(19) United States
(12) Patent Application Publication
(10) Pub. No.: US 2004/0088011 A1
(43) Pub. Date: May 6, 2004

(54) Defibrillation circuit that can compensate for a variation in a patient parameter and related defibrillator and method

(75) Inventors: David Ernest Snyder, Bainbridge Island, WA (US); Thomas Dean Lyster, Bothell, WA (US)

Correspondence Address: PHILIPS INTELLECTUAL PROPERTY & STANDARDS, P.O. BOX 3001, BRIARCLIFF MANOR, NY, 10510, US

(73) Assignee: Koninklijke Philips Electronics N.V.

(21) Appl. No.: 10/286,037

(22) Filed: Oct. 31, 2002

(51) Int. Cl. A61N 1/39

(52) U.S. Cl. 607/5

(57) ABSTRACT

A defibrillation circuit generates a defibrillation pulse and includes a patient-parameter compensator that causes the pulse to have a predetermined characteristic regardless of the value of a patient parameter. For example, the circuit can generate defibrillation pulses that have a desired shape, decay rate, voltage level, current level, and/or energy level regardless of the patient impedance. Consequently, a defibrillator that includes the circuit is likely to be more effective than prior defibrillators in restoring normal heart rhythms to patients having an atypical value for a parameter such as impedance.

### FIELD OF THE INVENTION

The invention relates generally to a medical device such as an external defibrillator, and more particularly to a defibrillation circuit that can compensate for a parameter, such as the impedance, of a patient. Such compensation allows the circuit to generate a defibrillation pulse having a desired characteristic regardless of the value of the patient parameter.

### BACKGROUND OF THE INVENTION

AEDs have saved many lives in non-hospital settings, and, as a result of advances in AED technology, the number of lives saved per year is rising. An AED is a battery-operated device that analyzes a patient's heart rhythm, and, if appropriate, administers an electrical shock (automated) or instructs an operator to administer an electrical shock (semi-automated) to the patient via electrode pads. For example, such a shock can often revive a patient who is experiencing ventricular fibrillation (VF).

As discussed below in conjunction with FIGS. 1 and 2, an AED typically generates one or more shocks, i.e., defibrillation pulses, that ideally will have one or more characteristics that the AED manufacturer has determined to be effective in restoring a normal heart rhythm to a patient. Examples of these characteristics include the shape, duration, energy, voltage, and current levels of the pulse, and the time constant according to which the pulse decays.

Unfortunately, a variation in one or more patient parameters may alter one or more characteristics of the defibrillation pulses in an undesired manner. For example, the impedance of the human body may affect the time constant according to which a defibrillation pulse decays, and this impedance typically varies from patient to patient. Consequently, if the patient impedance differs from an anticipated value, then it may alter one or more of the pulse characteristics in a manner that degrades the effectiveness of the defibrillation pulse.

FIG. 1 is a schematic diagram of a conventional defibrillation circuit 10, electrode pads 12a and 12b, and a patient that is modeled as an impedance Rp. The circuit 10 includes a capacitor 14 for storing pulse energy, a high-voltage generator 16 for charging the capacitor 14, a protection resistor RL for limiting the short-circuit current through the pads 12a and 12b, and a switch 18 such as a bridge for coupling the capacitor 14 to the patient via the pads 12.

FIG. 2 is a timing diagram of a Biphasic Truncated Exponential (BTE) defibrillation pulse 20 (solid line) having desired characteristics, a BTE pulse 22 (short-dash line) having undesired characteristics caused by a higher-than-expected patient impedance Rp, and a BTE pulse 24 (long-dash line) having undesired characteristics caused by a lower-than-expected Rp. Each pulse 20, 22, and 24 has a positive phase of duration TP and a negative phase of duration TN. Each phase is measured across Rp, and the shape, energy level, voltage level, current level, and time constant according to which the phases decay all depend on Rp. Specifically, assuming that the switch 18 (FIG. 1) has negligible impedance when closed, the voltage and current levels are respectively given by the voltage and current dividers formed by Rp and RL, the RC time constant is defined by the capacitance C₁₄ of the capacitor 14, Rp, and RL, the shape, i.e., the curve of exponential decay, is defined by the time constant, and the energy level is partially defined by the current through Rp. Consequently, for a given capacitance C of the capacitor 14 and a given voltage V across the capacitor, the voltage applied to the patient and the RC time constant increase as Rp increases, and the current level decreases as Rp increases. Furthermore, for given values for C, V, and phase durations Tp and Tn, the energy level delivered to the patient decreases as Rp increases.

Referring to FIGS. 1 and 2, the defibrillation circuit 10 generates one of the undesired BTE pulses 22 and 24 if the patient impedance Rp does not have an expected value. After a rescuer (not shown in FIGS. 1 and 2) attaches the pads 12a and 12b to the patient (represented by Rp) and while the switch 18 is open, the generator 16 charges the capacitor 14 to a voltage level Vc that is typically in the range of 1000 Volts (V)-3000 V. The manufacturer selects the capacitance C of the capacitor 14 and the voltage level Vc by assuming a typical value for Rp such as 85 Ω. After the capacitor 14 is charged, the switch 18 closes to deliver the pulse to the patient via the pads 12a and 12b. If Rp equals or approximately equals the assumed value of 85 Ω, then the positive phase of the BTE pulse 20 having the desired characteristics is delivered to the patient. If, however, Rp is greater than 85 Ω, then the positive phase of the BTE pulse 22 having a flatter-than-desired decay slope, higher-than-desired voltage level, and lower-than-desired current level is delivered to the patient. Conversely, if Rp is less than 85 Ω, then the positive phase of the BTE pulse 24 having a steeper-than-desired decay slope, lower-than-desired voltage level, and higher-than-desired current level is delivered to the patient. The switch 18 then opens for a wait period Tw, and closes again with a reversed polarity to generate the corresponding negative phase of the BTE pulse 20, 22, or 24.

Although BTE pulses are discussed above in conjunction with FIGS. 1 and 2, variations in a patient parameter such as the patient impedance Rp can also cause other types of defibrillation pulses to have undesired characteristics. Examples of other types of defibrillation pulses include but are not limited to damped sinusoid, Monophasic Truncated Exponential (MTE), rectilinear biphasic, and multiphasic defibrillation pulses.

Consequently, a need exists for a defibrillation circuit that can generate a defibrillation pulse having one or more desired characteristics regardless of the value of a patient parameter.

### SUMMARY OF THE INVENTION

In one embodiment of the invention, a defibrillation circuit includes an element for storing pulse energy and a patient-parameter compensator for causing a defibrillation pulse to have a predetermined characteristic regardless of the value of a patient parameter.

Such a defibrillation circuit can, therefore, generate a defibrillation pulse having a desired characteristic regardless of the value of one or more patient parameters. For example, the circuit can generate a defibrillation pulse that decays according to a desired time constant regardless of the value of the patient impedance. Consequently, a defibrillator that includes this circuit is likely to be more effective than prior defibrillators in restoring normal heart rhythms to patients having an a typical value for a parameter such as impedance.

### BRIEF DESCRIPTION OF THE DRAWINGS

FIG. 1 is a schematic diagram of a conventional defibrillation circuit for generating a defibrillation pulse.

FIG. 2 is diagram of three BTE defibrillation pulses that the defibrillation circuit of FIG. 1 respectively generates for a patient having a typical impedance, a higher-than-typical impedance, and a lower-than-typical patient impedance.

FIG. 3 is a schematic diagram of a defibrillation circuit according to an embodiment of the invention.

FIG. 4 is a schematic diagram of a defibrillation circuit according to another embodiment of the invention.

FIG. 5 is a view of an AED system having an AED that incorporates the defibrillation circuit of FIG. 3 or FIG. 4 according to an embodiment of the invention.

FIG. 6 is a block diagram of an AED circuit that the AED of FIG. 5 incorporates according to an embodiment of the invention.

### DESCRIPTION OF THE PREFERRED EMBODIMENTS

The following discussion is presented to enable a person skilled in the art to make and use the invention. Various modifications to the embodiments will be readily apparent to those skilled in the art, and the generic principles herein may be applied to other embodiments and applications without departing from the spirit and scope of the present invention as defined by the appended claims. Thus, the present invention is not intended to be limited to the embodiments shown, but is to be accorded the widest scope consistent with the principles and features disclosed herein.

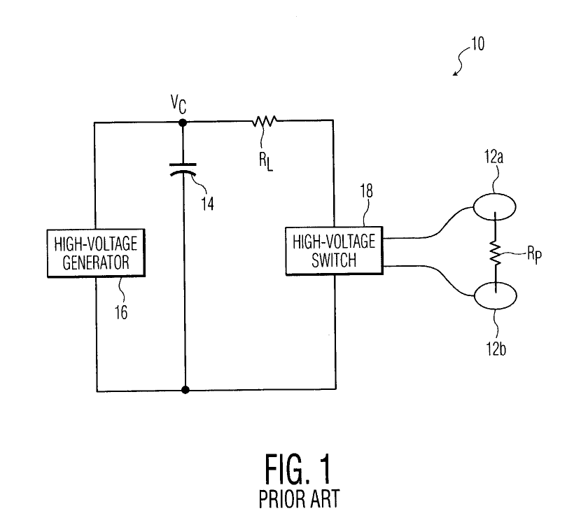

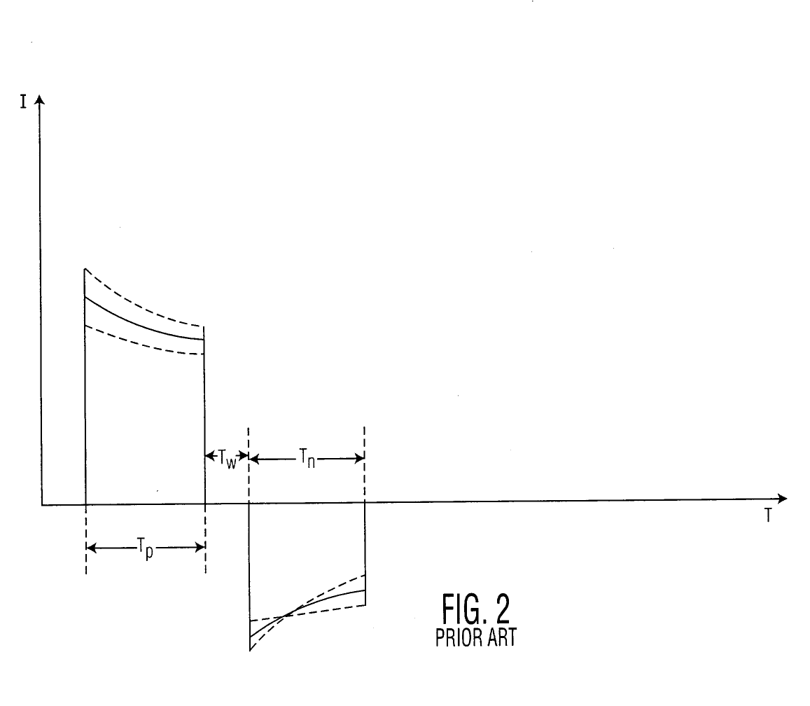

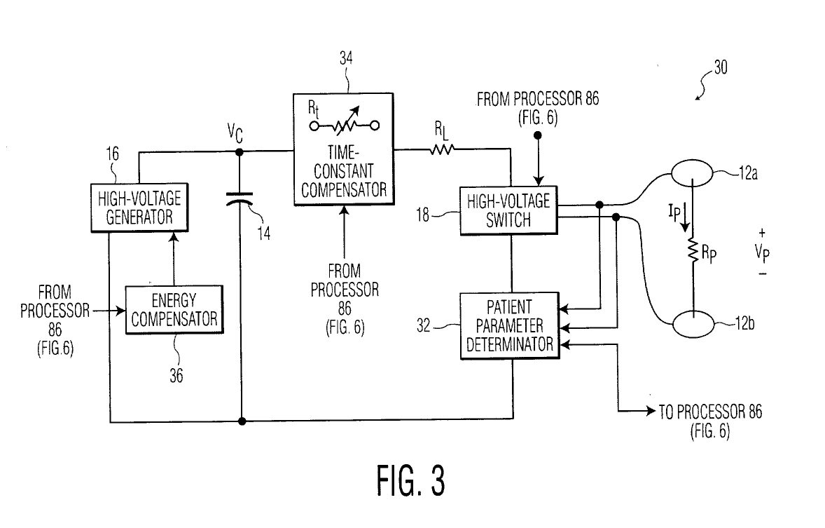

FIG. 3 is a schematic diagram of a defibrillation circuit 30 that can, according to an embodiment of the invention, generate a defibrillation pulse having a predetermined characteristic regardless of the value of a patient parameter, and where like numbers reference like components with respect to the defibrillation circuit 10 of FIG. 1. In the embodiments discussed below, the circuit 30 generates a BTE pulse such as the BTE pulse 20 of FIG. 2, although the circuit 30 can generate MTE and multiphasic pulses, and can be modified to generate other types of defibrillation pulses.

In addition to the capacitor 14, generator 16, switch 18, and limiting resistor RL, the circuit 30 includes a patient-parameter determiner 32 for measuring one or more patient parameters, a time-constant compensator 34 for allowing selection of a predetermined RC₁₄ time constant for the circuit 30, and an energy compensator 36 for allowing selection of a predetermined voltage level, current level, or energy level for the pulse. Consequently, the compensators 34 and 36 allow the circuit 30 to generate a defibrillation pulse having one or more desired characteristics even if a patient parameter such as the impedance Rp varies from an expected value.

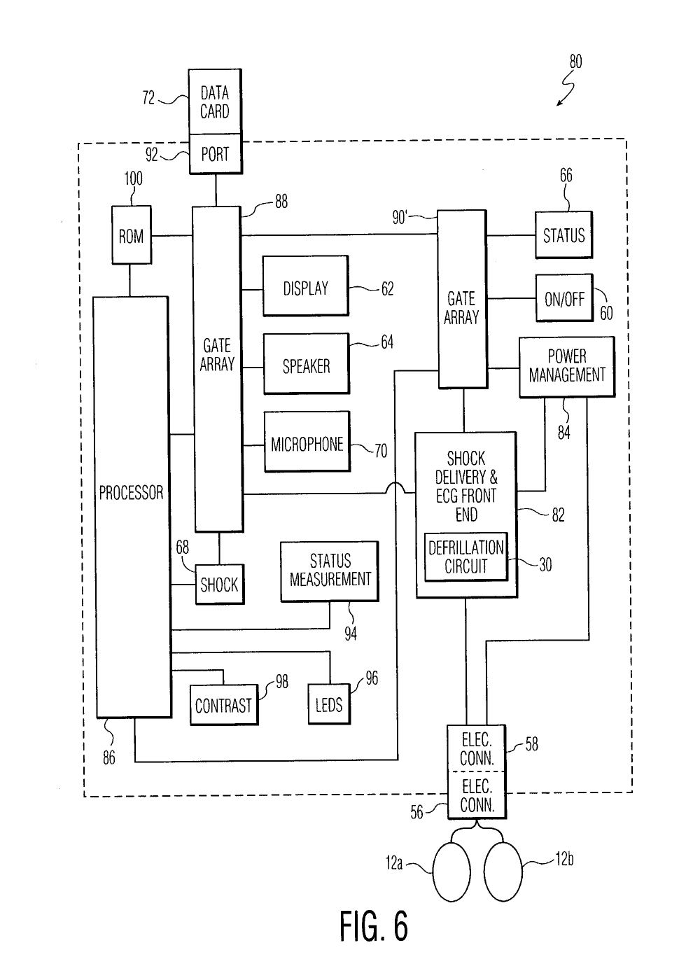

Still referring to FIG. 3, the determiner 32 measures a current through and a voltage across the electrodes 12a and 12b while the electrodes are attached to the patient, and a processor (FIG. 6) calculates the patient impedance Rp from these measurements. The determiner 32 may also measure other quantities such as the patient's temperature, and the processor may calculate other patient parameters from these quantities. Because circuits for measuring the voltage across and current through the electrodes 12a and 12b are known, a detailed discussion of such circuits is omitted.

The time-constant compensator 34 adds a selectable resistance Rt in series with the patient resistance Rp such that:

Rt=R−RL−Rp (1)

where R is a predetermined resistance value that gives a desired RC time constant and the value of Rt depends on the value of Rp as determined by the processor (FIG. 6). For example, assume that RL=10 Ω, the anticipated range of the patient impedance is 30 Ω≦Rp≦140 Ω, and the desired R=200 Ω. Therefore, if the processor determines that Rp=35 Ω, then it causes the compensator 34 to set Rt=155 Ω such that 35 Ω+10 Ω+155 Ω=200 Ω. Similarly, if the processor determines that Rp=120 Ω, then it causes the compensator 34 to set Rt=70 Ω such that 120 Ω+10 Ω+70 Ω=200 Ω. Consequently, the RC time constant remains at a predetermined value regardless of the value of Rp. And because RC remains at a predetermined value, the slope of decay, and thus the shape, of the defibrillation pulses remains constant. The compensator 34 can be implemented with a conventional resistor network (not shown) or any other conventional circuit that allows the processor to select a desired value for Rt. Depending on the topology of such a network/circuit, the processor may be unable to select the exact desired value for Rt, and thus may select the closest value of Rt available to approximate the desired RC time constant. For example, the network/circuit may be a bank of resistors that can be coupled together in different configurations to provide a finite number of values for Rt. Because such networks/circuits can be conventional, they are not discussed in detail.

The energy compensator 36 allows one to select the level of the voltage Vc to which the generator 16 charges the capacitor 14 so as to set the voltage, current, or energy of the defibrillation pulses at predetermined levels regardless of the value of Rp. For example, the peak voltage level Vp across the patient is given by the following equation:

Vp=VcRp/R (2)

Therefore, once the processor (FIG. 6) determines the value of Rp, it can calculate the value of Vc needed to obtain the desired value of Vp and set the compensator 36 accordingly. Similarly, the peak current Ip through the patient is given by the following equation:

Ip=Vc/R (3)

Because R is a predetermined value such as 200 Ω, the processor can at any time calculate the value of Vc needed to give the desired value of Ip and set the compensator 36 accordingly. Furthermore, the energy E in Joules delivered by the pulse to the patient is given by the following equation:

$$E = \frac{R p {V}^{2} C}{2 R} [ 1 - {ⅇ}^{\frac{2 ( Tp + Tn )}{R C}} ] \qquad ( 4 )$$

Therefore, once the processor calculates the value of Rp, it can determine the value of Vc necessary to give the desired energy E and set the compensator 36 accordingly. Alternatively, the processor can adjust one or both of the durations Tp and Tn to obtain the desired energy E by adjusting the time that the switch 18 is closed. Or, the processor can adjust Vc and one or both of the durations Tp and Tn to obtain the desired energy E. The compensator 36 can be implemented with a conventional comparator circuit (not shown) or any other circuit that allows the processor to select a desired value of Vc.

Still referring to FIG. 3, the defibrillation circuit 30 operates as follows. First, the determiner 32 measures the current through and voltage across the patient and provides these measurements to the processor (FIG. 6), which calculates Rp therefrom. This measurement and calculation may occur before the BTE pulse using a test current or voltage, or during an initial portion of the BTE pulse. Next, the processor calculates the values of Rt and Vc based on Rp and the desired pulse characteristics, which are typically preprogrammed into the processor memory (FIG. 6), and sets the time-constant and energy compensators 34 and 36 accordingly. Then, the generator 16 charges the capacitor 14 to Vc, and the processor closes the switch 18 to deliver the positive phase (Tp) of the pulse. Next, the processor opens the switch 18 for the predetermined wait time Tw, and then closes it again to deliver the negative phase (Tn) of the BTE pulse. Under control of the processor, the circuit 30 may deliver additional BTE pulses that have the same or different characteristics as the initial BTE pulse. To change the pulse characteristics, the processor can cause the compensators 34 and 36 to change the value of Rt and/or Vc based on the previously calculated value of Rp. Alternatively, the determiner 32 can take one or more measurements of the current through and voltage across the patient, and the processor can recalculate Rp based on these new measurements and change the value of Rt and/or Vc based on the recalculated Rp. Or the processor may recalculate Rp and/or Vc based on the characteristics of the previous pulse or pulses.

Other embodiments of the defibrillator circuit 30 are contemplated. For example, either one of the time-constant and energy compensators 34 and 36 may be omitted from the circuit 30. Furthermore, the circuit 30 may include a filter, such as an inductor (not shown) situated between the capacitor 14 and the patient Rp, to modify the shape and/or other characteristics of the defibrillation pulse. Moreover, the circuit 30 may modify the characteristics of one phase of a muliphasic defibrillation pulse differently than it modifies the characteristics of another phase by reconfiguring the filter or other circuitry between phases.

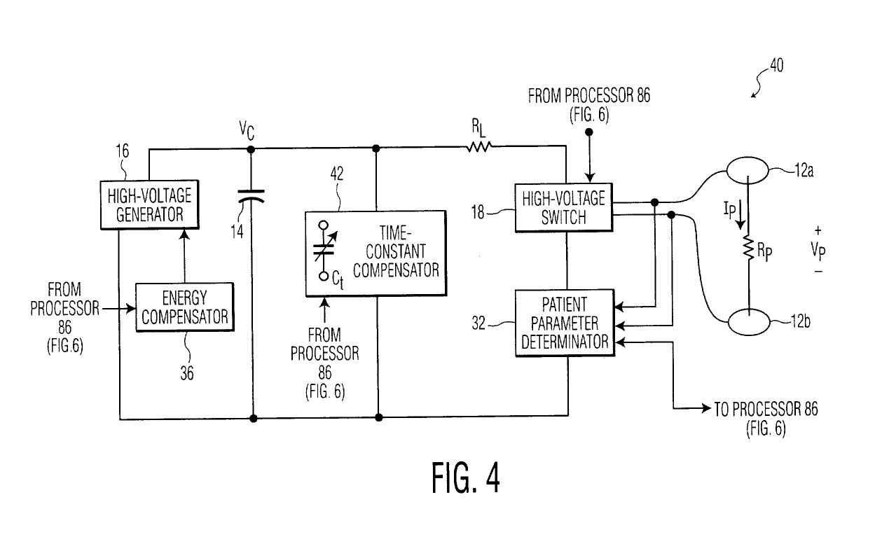

FIG. 4 is a schematic diagram of a defibrillation circuit 40 that includes a time-constant compensator 42 according to another embodiment of the invention, and where like numbers reference like components with respect to the defibrillation circuit 30 of FIG. 3. The circuit 40 is similar to the circuit 30 except that unlike the time-constant compensator 34 of the circuit 30, the time-constant compensator 42 is in parallel, not in series, with the capacitor 14. Like the circuit 30, the circuit 40 can generate a defibrillation pulse having a predetermined characteristic regardless of the value of a patient parameter such as the patient impedance. An advantage of the circuit 40 is that because it lacks the series resistance Rt, it often dissipates less energy than the circuit 30.

The time-constant compensator 42 adds a capacitance Ct in parallel with the capacitor 14 such that:

Ct=C−C₁₄ (5)

where C is the total capacitance needed to give the desired RC time constant and the value of Ct depends on the value of Rp determined by the processor (FIG. 6). For example, assume that RL=10 Ω, the anticipated range of the patient impedance is 30 Ω≦Rp≦140 Ω, C₁₄=50 μF, and the desired time constant is 5 milliseconds (ms). Therefore, if the processor determines that Rp=75 Ω, then it causes the compensator 42 to set Ct=9 μF such that (C=50 μF+9 μF)×(R=75 Ω+10 Ω)=5 ms. Consequently, the processor can set the RC time constant to a desired value regardless of the value of Rp. And because the processor can set RC, the processor can set the slope of decay, and thus the shape, of the defibrillation pulses. The compensator 42 can be implemented with a conventional capacitor network or any other circuit that allows the processor to select a desired value for Ct. Depending on the topology of such a network/circuit, the processor may be unable to select the exact desired value for Ct, and thus may select the closest value of Ct available to approximate the desired RC time constant. For example, the network/circuit may be a bank of capacitors that can be coupled together in different configurations to provide a finite number of values for Ct. Because such networks/circuits can be conventional, they are not discussed in detail.

Still referring to FIG. 4, the processor (FIG. 6) can set Vc via the energy compensator 36 as discussed above in conjunction with FIG. 3 to obtain desired values for Vp, Ip, and E.

Other embodiments of the defibrillator circuit 40 are contemplated. For example, the circuit 40 may include both the compensator 42 and the compensator 32 of the circuit 30. Also contemplated are embodiments that are similar to the other embodiments of the defibrillator circuit 30 discussed above in conjunction with FIG. 3.

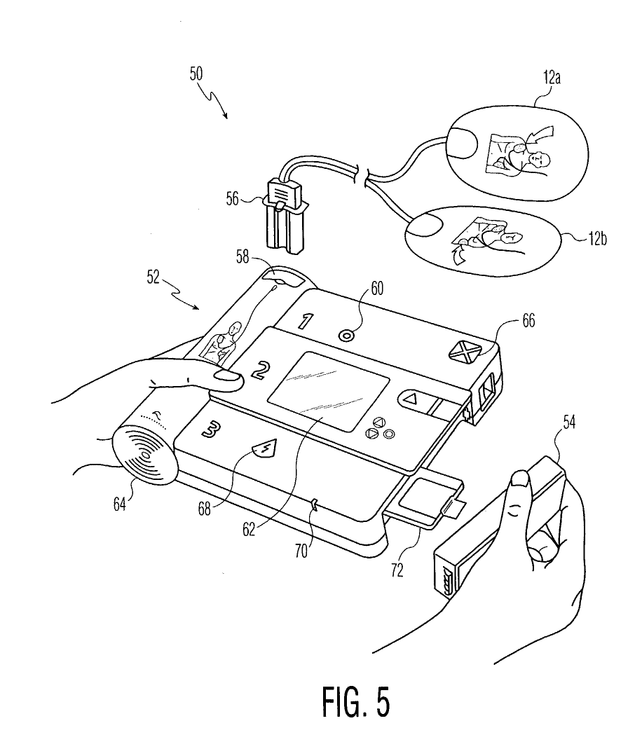

FIG. 5 is a view of a conventional AED system 50, which includes an AED 52 that incorporates the defibrillation circuit 30 (FIG. 3) or the defibrillation circuit 40 (FIG. 4) according to an embodiment of the invention. The system 50 also includes the electrode pads 12a and 12b for providing the shock to the patient (not shown), and a battery 54. A connector 56 couples the electrode pads 12a and 12b to a receptacle 58 of the AED 52.

The AED 52 includes a main on/off key switch 60, a display 62 for displaying operator instructions, cardiac waveforms, or other information, a speaker 64 for providing audible operator instructions or other information, an AED status indicator 66, and a shock button 68, which the operator (hands shown) presses to deliver a shock to the patient (not shown). The AED 52 may also include a microphone 70 for recording the operator's voice and other audible sounds that occur during the rescue, and a data card 72 for storing these sounds along with the patient's ECG and a record of AED events for later study.

Still referring to FIG. 5, during an emergency where it is determined that the patient (not shown) may need a shock, the operator retrieves the AED 52 and installs the battery 54 if it is not already installed. Next, the operator removes the electrode pads 12a and 12b from their protective package (not shown) and inserts the connector 56 into the receptacle 58. Then, the operator turns the on/off switch 60 to the “on” position to activate the AED 52. Following the instructions displayed on the display 62 or “spoken” via the speaker 64, the operator places the electrode pads 12a and 12b on the patient in the respective positions shown in the pictures on the pads and on the AED 52. After the operator places the electrode pads 12a and 12b on the patient, the AED 52 analyzes the patient's ECG to determine whether the patient is suffering from a shockable heart rhythm. If the AED 52 determines that the patient is suffering from a shockable heart rhythm, then it instructs the operator to depress the shock button 68 to deliver a shock to the patient. Conversely, if the AED 52 determines that the patient is not suffering from a shockable heart rhythm, it informs the operator to seek appropriate non-shock treatment for the patient and often disables the shock button 68 so that even if the operator presses the button 68, the AED 52 does not shock the patient.

Although described in conjunction with the AED 52, the defibrillation circuit 30 (FIG. 3) and the defibrillation circuit 40 (FIG. 4) may be incorporated by other types of external defibrillators.

FIG. 6 is a block diagram of an AED circuit 80, which the AED 52 of FIG. 5 can incorporate according to an embodiment of the invention. The circuit 80 includes a shock-delivery-and-ECG-front-end circuit 82 that includes the defibrillator circuit 30 (FIG. 3) or the defibrillator circuit 40 (FIG. 4). For example purposes, however, the circuit 82 is shown incorporating the circuit 30.

In addition to the shock-delivery-and-ECG-front-end circuit 82, the AED circuit 80 includes a power-management circuit 84, which interfaces with a processor 86 via a gate array 88. Under the control of the processor 86, the power-management circuit 84 distributes power from the battery 54 (FIG. 5) to the other subcircuits of the circuit 80. In addition, the processor 86 may monitor the voltage across the battery 54 via the power-management circuit 84 and generate an alarm via the display 62, speaker 64, or other means to indicate that the battery 54 needs to be replaced.

During treatment of the patient (not shown), the shock-delivery-and-ECG-front-end circuit 82, samples the patient's ECG to determine if the patient is suffering from a shockable heart arrhythmia. The processor 86 receives the samples from the circuit 82 via the gate array 88 and analyzes them. If analysis indicates that the patient is suffering from a shockable heart rhythm, then the processor 86 instructs the circuit 82 via the gate array 88 to enable delivery of a shock to the patient when an operator (FIG. 5) presses the shock button 68. Conversely, if analysis indicates that the patient is not suffering from a shockable heart rhythm, then the processor 86 effectively disables the shock button 68 by preventing the circuit 82 from delivering a shock to the patient if/when the operator presses the shock button 68.

Still referring to FIG. 6, the on/off switch 60 turns the AED circuit 80 “on” and “off” and a gate array 90 interfaces the power-management circuit 84, the on/off switch 60, and the status indicator 66 to the shock-delivery-and-ECG-front-end circuit 82, the processor 86, and the gate array 88.

The circuit 80 also includes the display 62, which presents information to an operator, the speaker 64, which may provide audio instructions to the operator, and the microphone 70, which may record the operator's voice and other audible sounds. The data card 72 is connected to the gate array 88 via a port 92, and may store the operator's voice and other sounds along with the patient's ECG and a record of AED events for later study.

A status-measurement circuit 94 provides the status of the other circuits of the AED circuit 80 to the processor 86, and LEDs 96 and the status indicator 66 provide information to the operator (FIG. 5) such as whether the processor 86 has enabled the shock-delivery-and-ECG-front-end circuit 82 to deliver a shock to the patient (not shown). A contrast button 98 allows the operator to control the contrast of the display screen 62 if present, and a memory such as a read only memory (ROM) 100 stores programming information for the processor 86 and the gate arrays 88 and 90. The ROM 100 may also store the desired characteristics for the defibrillator pulses generated by the defibrillator circuit 30.

The AED circuit 80 and other similar AED circuits that may incorporate the shock-delivery-and-ECG-front-end circuit 82 are discussed in the following references, which are incorporated by reference: U.S. Pat. No. 5,836,993, U.S. Pat. No. 5,735,879 entitled ELECTROTHERAPY METHOD AND APPARATUS, U.S. Pat. No. 5,607,454 entitled ELECTROTHERAPY METHOD AND APPARATUS, and U.S. Pat. No. 5,879,374 entitled DEFIBRILLATOR WITH SELF-TEST FEATURES.

What is claimed is:

1. A circuit for defibrillating a patient with a defibrillation pulse, the patient having a parameter, the circuit comprising:
an energy-storage element; and
a parameter compensator coupled to the energy-storage element and operable to cause the pulse to have a predetermined characteristic regardless of the patient parameter's value.

2. The circuit of claim 1, further comprising:
wherein the energy-storage element comprises a capacitor; and
a switch operable to couple the capacitor to the patient.

3. The circuit of claim 1, further comprising a parameter determiner operable to measure a quantity from which the patient parameter can be calculated.

4. The circuit of claim 1, further comprising a current-limiting element that is in series with the energy-storage element.

5. The circuit of claim 1 wherein the parameter compensator is operable to cause the defibrillation pulse to decay according to a predetermined time constant regardless of the patient parameter's value.

6. A circuit for defibrillating a patient having an impedance, the circuit comprising:
a storage element operable to store defibrillation energy and to define a time constant with the patient impedance; and
an impedance compensator coupled to the storage element and operable to cause the time constant to have a predetermined value.

7. The circuit of claim 6 wherein the storage element comprises a capacitor.

8. The circuit of claim 6 wherein the impedance compensator includes an adjustable impedance.

9. The circuit of claim 6, further comprising an impedance determiner operable to measure a current through the patient.

10. The circuit of claim 6, further comprising an impedance determiner operable to measure a voltage across the patient.

11. The circuit of claim 6, further comprising a generator operable to charge the storage element with the defibrillation energy.

12. The circuit of claim 6, further comprising a defibrillation-energy-delivery switch operable to couple the storage element to the patient.

13. The circuit of claim 6 wherein the impedance compensator comprises an adjustable resistor operable to be coupled between the storage element and the patient.

14. The circuit of claim 6 wherein the impedance compensator comprises an adjustable capacitor in electrical parallel with the storage element.

15. The circuit of claim 6 wherein the impedance compensator comprises a resistor network operable to be coupled between the storage element and the patient and to provide selectable resistance values.

16. The circuit of claim 6 wherein the impedance compensator comprises a capacitor network in electrical parallel with the storage element and operable to provide selectable capacitance values.

17. A circuit for defibrillating a patient having an impedance, the circuit comprising a storage element that is operable to:
store defibrillation energy;
define a time constant with the patient impedance; and
allow adjustment of the time constant.

18. The circuit of claim 17 wherein the storage element comprises an adjustable capacitor.

19. The circuit of claim 17 wherein the storage element comprises a capacitor network operable to provide selectable capacitance values.

20. A circuit for defibrillating a patient having a parameter, the circuit comprising:
a storage element operable to store defibrillation energy; and
an energy compensator coupled to the storage element and operable to control the level of defibrillation energy stored in the storage element based on the patient parameter's value.

21. The circuit of claim 20 wherein the patient parameter comprises an impedance of the patient.

22. The circuit of claim 20, further comprising:
wherein the patient parameter comprises a patient impedance having a value; and
an impedance compensator coupled to the storage element and operable to cause a defibrillation pulse formed from the defibrillation energy to decay according to a predetermined time constant regardless of the value of the patient impedance.

23. The circuit of claim 20, wherein the energy compensator is operable to cause a defibrillation pulse generated from the defibrillation energy to have a predetermined peak voltage regardless of the patient parameter's value.

24. The circuit of claim 20, wherein the energy compensator is operable to cause a defibrillation pulse generated from the defibrillation energy to have a predetermined peak current regardless of the patient parameter's value.

25. A defibrillator, comprising:
a control circuit; and
a shock-delivery circuit coupled to the control circuit and operable to generate a defibrillation pulse for a patient having a parameter, the shock delivery circuit comprising,
an energy-storage element, and
a parameter compensator coupled to the energy-storage element and operable to cause the pulse to have a predetermined characteristic regardless of the patient parameter's value.

26. The defibrillator of claim 25 wherein the control circuit comprises a processor.

27. A method, comprising:
determining a value of a parameter of a patient; and
providing a defibrillation pulse to the patient, the pulse having a predetermined characteristic regardless of the parameter's value.

28. The method of claim 27 wherein the predetermined characteristic comprises a predetermined time constant according to which the pulse decays.

29. The method of claim 27 wherein the predetermined characteristic comprises a predetermined peak-voltage level of the pulse.

30. The method of claim 27 wherein the predetermined characteristic comprises a predetermined peak-current level of the pulse.

31. The method of claim 27 wherein the predetermined characteristic comprises a predetermined energy-level delivered to the patient by the pulse.

32. The method of claim 27 wherein the parameter comprises an impedance of the patient.

33. A method, comprising:
determining a parameter of a patient; and
combining a compensation parameter with the patient parameter to generate a combined parameter having a predetermined value.

34. The method of claim 33 wherein:
the patient parameter comprises a patient impedance;
the compensation parameter comprises a compensation impedance; and
combining the compensation and patient parameters comprises combining the compensation and patient impedances to generate a combined impedance having a predetermined value.
